(19) United States
(12) Patent Application Publication
(10) Pub. No.: US 2024/0042065 A1
(43) Pub. Date: Feb. 8, 2024

(54) THERANOSTIC AGENTS

(71) Applicant: CuhaWijay Sathiyajith, Westmead (AU)

(72) Inventor: CuhaWijay Sathiyajith, Westmead (AU)

(21) Appl. No.: 18/111,153

(22) Filed: Feb. 17, 2023

(30) Foreign Application Priority Data
Jul. 28, 2022 (AU) 2022902128

(51) Int. Cl. A61K 51/04; A61P 35/00

(52) U.S. Cl. CPC A61K 51/0497; A61P 35/00

(57) ABSTRACT

Disclosed herein is the use of compounds having the formulae (I) and (II), as described herein, as theranostic agents.

### TECHNICAL FIELD

The present invention relates to compounds that are useful as theranostic agents.

### BACKGROUND ART

Medical imaging is a technique that is used to produce images of the interior of a patient's body for clinical analysis. Imaging agents are often used in medical imaging procedures, generally resulting in an enhancement of the resultant images. For example, imaging agents may preferentially target disease cells (particularly cancer cells), resulting in images that highlight the prevalence and location of such cells in a patient. So-called multimodal imaging agents are agents that have properties which enable them to be used with two or more imaging techniques. Such multimodal functionality enables the results from two or more different imaging techniques to be combined in order to improve the usefulness of the images.

Theranostic agents have both diagnostic and therapeutic functionality, where a single substance can provide for both the imaging and treatment of a disease. Such agents can be used for simultaneous targeted drug delivery and release and diagnosis, including monitoring disease progression and response to therapy. Theranostic agents can be used to provide a level of personalised medicine which was previously not possible, especially in the oncology and imaging fields.

It would be advantageous to provide additional theranostic agents to those presently available.

### SUMMARY OF INVENTION

In a first aspect, the present invention provides the use of a compound having the formula (I):

M-L-X (I)

as a theranostic agent, wherein:
M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II);
L is an aminopolycarboxylic acid ligand; and
X is a chromophoric substituent on L that has a therapeutic activity.

In a second aspect, the present invention provides the use of a compound having the formula (II):

M-L (II)

as a theranostic agent, wherein:
M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II); and
L is selected from the group consisting of:

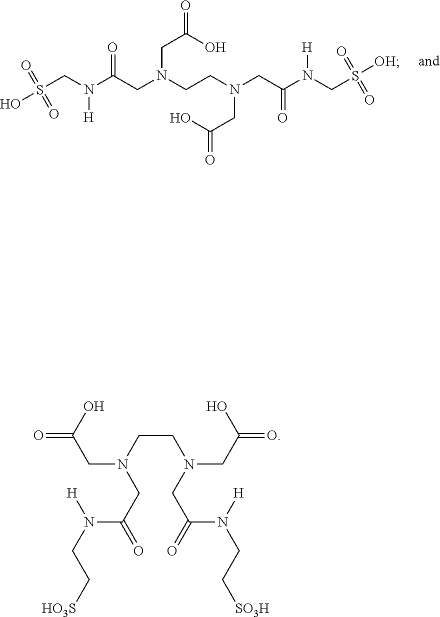 [SMILES: O=C(O)CN(CCN(CC(=O)O)CC(=O)NCS(=O)(=O)O)CC(=O)NCS(=O)(=O)O ; O=C(O)CN(CCN(CC(=O)O)CC(=O)NCCS(=O)(=O)O)CC(=O)NCCS(=O)(=O)O]

In a third aspect, the present invention provides the use of a compound having the formula (I) or formula (II), for the manufacture of a medicament for combined use as an imaging agent and for the treatment of cancer.

In a fourth aspect, the present invention provides a compound having the formula (I) or formula (II) for the treatment of cancer.

In a fifth aspect, the present invention provides a method for treating cancer in a patient. The method comprises the step of administering a compound having the formula (I) or formula (II) to the patient. The method may further comprise imaging the cancer after administration of the compound.

The inventor has discovered that metal complexes having the formula (I) and formula (II) are not only useful as imaging agents (some of the compounds of formula (I) being multi-modal imaging agents), but they are also expected to have therapeutic effect when administered to a patient. The experiments conducted or commissioned by the inventor which lead them to make this prediction of theranostic activity will be described in further detail below.

In some embodiments, the substituent X may have an anticancer activity. The inventor predicts that the substituent X may have anticancer activity in relation to one or more of the following cancers: lung cancer, non-small cell lung cancer, breast cancer, bladder cancer, blood cancer, gastric cancer, ovarian cancer, liver cancer, pancreatic cancer, testicular cancer, prostate cancer, brain cancer, head/neck cancers and neuroendocrine tumours. Experiments that will confirm this prediction have been planned and are described below.

In some embodiments, the substituent X may, for example, be an Aurora kinase B inhibitor. Inhibition of Aurora kinase B has been reported to correlate with anticancer activity.

In some embodiments, the compound of formula (I) may comprise a plurality of the substituents X, wherein each substituent X is the same or different.

In some embodiments, the substituent X may be substituted at a carboxylic or an amine group of L, for example via an amide linkage, as described below. In some embodiments, substitution of L could be on a carbon, such as directly on one (or more) of the carbon atoms in the EDTA's ethylene diamine backbone or DTPA's diethylene triamine backbone.

In some embodiments, the substituent X may comprise a lumophoric or fluorophoric (or lumophoric and fluorophoric) moiety.

In some embodiments, the substituent X may comprise a moiety selected from the group consisting of: 4-amino methyl pyridine, 2-amino anthraquinone, sulphonamide, N-(2-aminoethyl)-1,8-napthalimide, 4-aminophenol, 9-amino acridine and 5-amino naphthalene-2-sulphonic acid.

In some embodiments, L may be a hexadentate or octadentate aminopolycarboxylic acid ligand. For example, in some embodiments, L may be selected from the group consisting of: ethylene diamine tetra acetic acid (EDTA), diethylene triamine penta acetic acid (DTPA), 1,4-bis(carboxymethyl)-6-[bis(carboxymethyl)]amino-6-methylperhydro-1,4-diazepine (AZTA), cyclohexylene dinitrilo tetra acetic acid (CDTA), 1,4,7,10-tetraazacyclododecane-1,4,7,10-tetraacetic acid (DOTA), nitrilotriacetic acid (NTA) and 1,2-propylenediaminetetraacetic acid (PDTA).

In some embodiments, L-X may be an EDTA-N,N″-bis(amide) ligand. In alternative embodiments, L-X may be an asymmetrically or symmetrically mono-, bis or tris-substituted DTPA.

In some embodiments, L-X may be selected from the group of ligands consisting of:

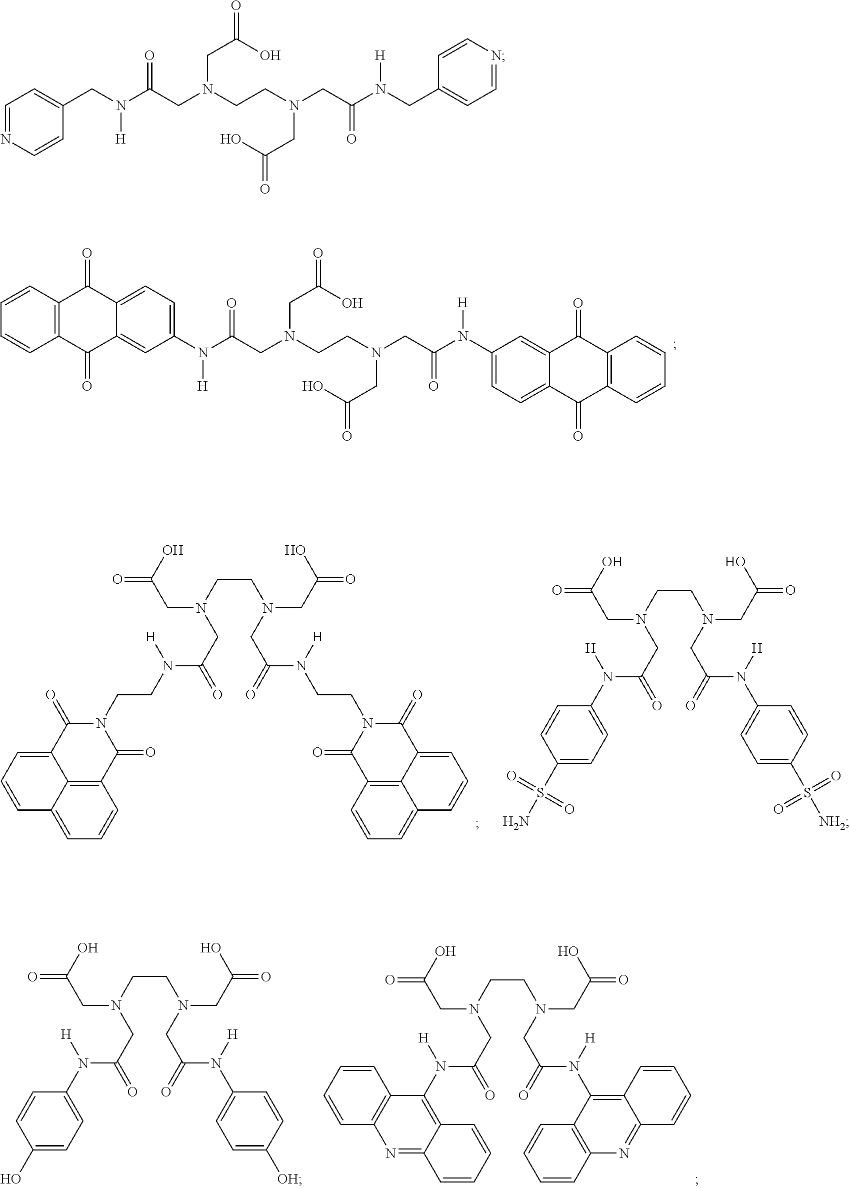

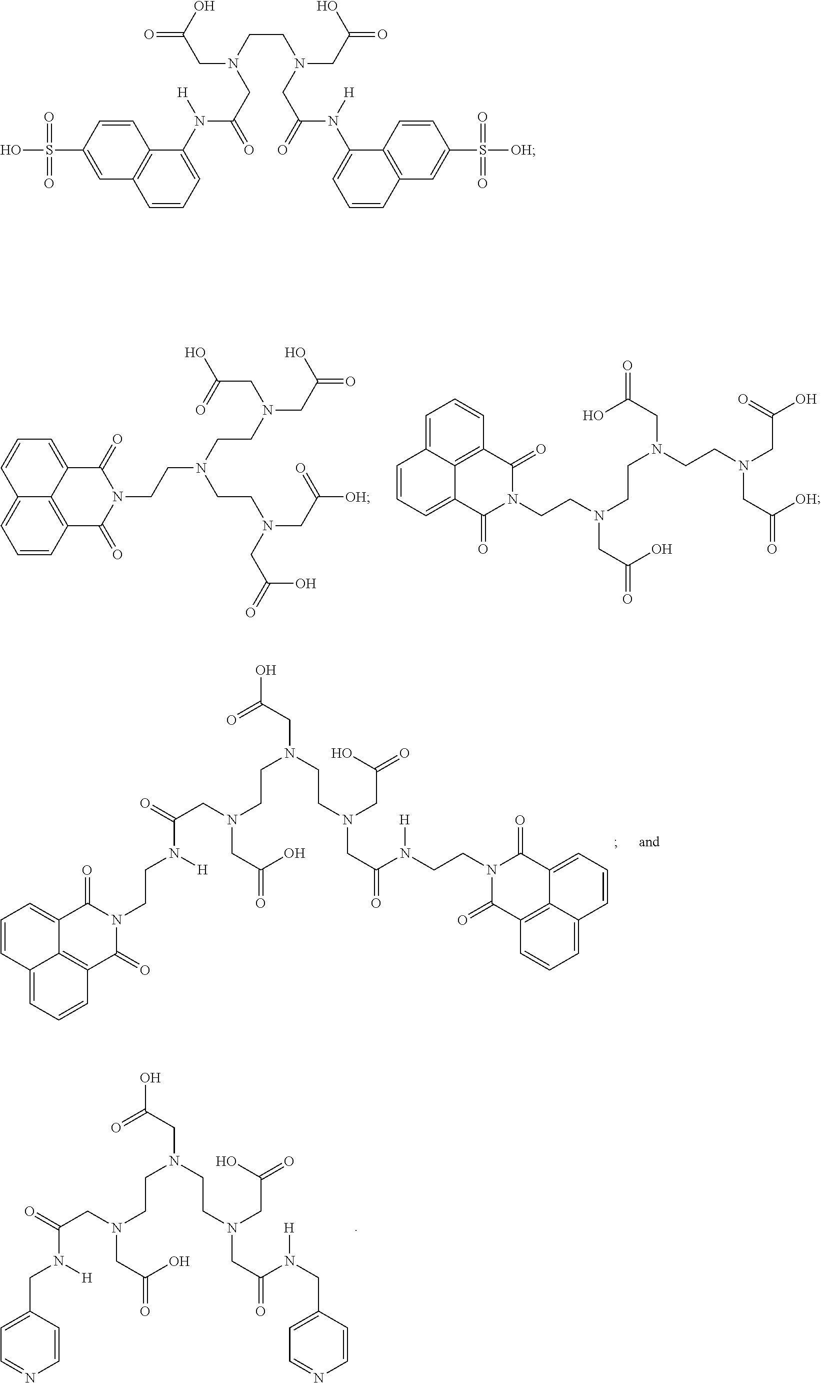

In some emoluments, the compound having the formula (I) may be a multi-modal imaging agent (e.g. a dual mode imaging agent). Such agents can overcome shortcomings associated with single mode imaging agents, such as a relative lack of specificity and lack of spatial resolution.

In some emoluments, for example, the compound having the formula (I) may be an imaging agent for two or more of the following imaging techniques: magnetic resonance imaging (MRI), positron emission tomography (PET), Dosimetry, Fluorescence lifetime imaging (FLI), Cerenkov luminescence imaging (CLI), computerized tomography (CT), single-photon emission computerized tomography (SPECT) and optical imaging (OI).

In some emoluments, radioactive isotopes of M may be used. For example, M may comprise a radioactive isotope of Mn(II), Cu(II), Zn(II), Gd(III), Ga(III), Zr(IV), Ac(III), Tc(IV) or Pb(II), specific examples of which are: ⁵¹Mn, ⁵²Mn, ⁶⁸Ga, ⁸⁹Zr, ²²⁵Ac, ⁹⁹Tc or ²¹²Pb. Such isotopes may even further enhance imaging or provide an additional therapeutic effect. In such embodiments, it will be appreciated that M may include mixtures of isotopes (e.g. a mixture of ⁵⁵Mn and ⁵²Mn and/or ⁵¹Mn), which may contribute to an enhanced imaging functionality.

The inventor also notes that isotopes of other metals (e.g. ²²⁵Ac, ¹⁷⁷Lu, ¹¹¹In, ⁹⁹Tc or ¹⁶⁶Ho) might also be incorporated into the present invention in order to provide an even further enhanced functionality.

It is to be understood that any features and embodiments described herein in detail in relation to a specific aspect of the invention are equally applicable to other aspects of the invention. Other aspects, features and advantages of the present invention will be described below.

### DETAILED DESCRIPTION OF THE INVENTION

As noted above, in its most general form, the present invention provides the use of compounds having the formula (I):

M-L-X (I)

as theranostic agents, wherein:
M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II);
L is an aminopolycarboxylic acid ligand (e.g. a hexadentate or octadentate aminopolycarboxylic acid ligand); and
X is a chromophoric substituent on L that has a therapeutic activity.

The use of a compound having the formula (II):

M-L (II)

as theranostic agents is also provided. In formula (II):
M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II); and
L is selected from the group consisting of:

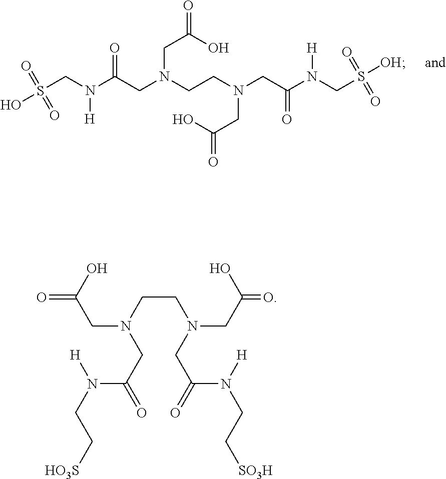 [SMILES: O=C(O)CN(CCN(CC(=O)O)CC(=O)NCS(=O)(=O)O)CC(=O)NCS(=O)(=O)O ; O=C(O)CN(CCN(CC(=O)O)CC(=O)NCCS(=O)(=O)O)CC(=O)NCCS(=O)(=O)O]

Also provided is the use of a compound having the formula (I) or formula (II), for the manufacture of a medicament for combined use as an imaging agent and for the treatment of cancer.

Also provided is a method for treating cancer in a patient, comprising administering a compound having the formula (I) or formula (II) to the patient. The method may further comprise imaging the cancer after administration of the compound having the formula (I) or formula (II).

As described above, the inventor has discovered that metal complexes having the formula (I) are not only useful as imaging agents (some of them being multi-modal imaging agents), but they are also likely to have therapeutic effect when administered to a patient because of the therapeutic activity of substituent X. In embodiments where the compound having formula (I) includes a substituent X that is an Aurora kinase B inhibitor, for example, the inventor predicts that the compound will have anticancer activity. The specific inhibition of Aurora B kinase has been demonstrated to result in anti-proliferative effects and cause regression in several animal models of human cancers, including breast, colon, lung, leukemia, prostate and thyroid. Inhibition is best affected by interfering with normal chromosomal alignment during cell division (particularly Mitosis phase) and overrides the mitotic spindle checkpoint inducing endoreduplication, leading to catastrophic mitosis culminating in apoptosis (cell death).

The in silico calculations described in further detail below support this potential therapeutic application of a compound falling within the scope of the present invention. The inventor predicts that the compounds having formula (I) will be therapeutically effective against cancers such as lung cancer, non-small cell lung cancer, breast cancer, bladder cancer, blood cancer, gastric cancer, ovarian cancer, liver cancer, pancreatic cancer, testicular cancer, prostate cancer, brain cancer, head/neck cancers and neuroendocrine tumours. Experiments that will confirm this prediction have been planned and are described below.

Similarly, the amino methyl sulphonic acid (AMSA) and taurine moieties on the EDTA bis(amide) ligands in the metal complexes having the formula (II) are expected to have therapeutic effect when administered to a patient, given their known properties. Specifically, AMSA has been demonstrated to exhibit anti-viral activity such as anti-influenza activity by modulating the intracellular redox potential thus preventing infection by suppression of reproduction of influenza strains (H1N1, H3N2 etc.). Further, AMSA, being a glycine analogue, through its antioxidant capacity could prevent the loss of activity of antioxidant enzymes closely associated with diabetes (e.g., SOD) induced by oxidative stress. Furthermore, the role of AMSA as hepatoprotective agent has been established in the LPS induced production TNF-α. Attendant therapeutic applications would be apparent to a person skilled in the art, and include as an aid in mobilizing endogenous antioxidant defense system and treatment of hepatic disorders such as chronic liver impairment (e.g. cirrhosis).

Taurine is a non-essential amino acid, which could act as a neuroprotective agent in the case of alcohol-induced conditions, specifically in the prevention of acute ethanol administration-induced apoptotic neurodegeneration of central nervous system. It also helps to mitigate effects of diabetes via antioxidant capacity. Again, attendant therapeutic applications would be apparent to a person skilled in the art.

Clinical applications the inventor expects compounds having the formulae (I) and (II) will have include.
Neuroimaging
Cardiac imaging (myocardial viability during cardiopathy)
Liver cancer imaging
Monitoring differentiation of stem cells during stem cell transplant therapy while monitoring neurodegenerative diseases
Postoperative care of chemotherapy patients
Ca²⁺ dependent abnormalities in aging, glaucomatous, and diabetic retinas
Calcium channel and potassium channel blockers, thus aiding in interventional neuroradiology
Oral nano theranostics delivery system
Intra retinal Ca(II) ion demand during the evaluation of Retinopathy of prematurity (ROP)
Translational neuroimaging—due to high relaxivity, receptor-targeted precise delivery low dose
pre-clinical neuroimaging
Contrast-enhanced detection of brain gliomas via monitoring cerebral blood volume
PrPC inhibition for post-recovery of stroke

Personalised medicine is touted as the future of patient management and health care, and medical imaging will be a key resource in achieving this objective. Imaging modalities such as Magnetic Resonance Imaging (MRI), computerized tomography (CT), Positron Emission Tomography (PET) and Optical Imaging (OI) are very useful, but all have shortcomings such as lack of specificity and lack of spatial resolution. A combination of multiple imaging techniques has been suggested to overcome these difficulties but this cannot be achieved by simple addition of two types of imaging agents, unless they have identical pharmacodynamic properties. Therefore, the necessity for the introduction of dual-purpose contrast agents or multimodal imaging probes has been justified and, in some embodiments of the present invention, compounds having the formula (I) provide for such multimodality.

As described herein, a number of the compounds having the formula (I) have been found by the inventor to be dual mode imaging agents. Further, the inventor expects that other compounds of formula (I) will be dual or multimodal imaging agents, given that their chemical structures include chromophoric groups and their chemical similarities to substances that have been found to have diagnostic functionality. Routine trials and experiments, such as those described herein, can be used to demonstrate this effect.

The compound of formula (I) might, for example, be an imaging agent for two or more of the following imaging techniques: magnetic resonance imaging (MRI), positron-emission tomography (PET), dosimetry, fluorescence lifetime imaging (FLI), computed tomography (CT), single-photon emission computed tomography (SPECT) and optical imaging (OD. In a specific embodiment, the invention may provide for precision oncology using a multimodal (e.g. MRI/OI, PET/OI and/or MRI/PET) anticancer theranostics agent for imaging, liver cancer, non-small cell lung cancer and blood cancer, for example.

In the compound of formula (I), M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II). The choice of metal will depend on the nature of the ligand L and the envisaged application of the theranostic agent (particularly the imaging technique). In some embodiments, the metal M may be a radioactive isotope of Mn(II), Cu(II), Zn(II), Gd(III), Ga(III), Zr(IV), Ac(III), Tc(IV) or Pb(II), such as ⁵¹Mn, ⁵²Mn, ⁶⁸Ga, ⁹⁹Tc, ²²⁵Ac, ⁸⁹Zr or ²¹²Pb, where such would provide any functional advantages in the context of the present invention. For example, compounds of formula (I) including ⁵⁵Mn may, in principle, result in a MRI/PET dual modal imaging agent. By using a mixture of ⁵⁵Mn (natural) with ⁵²Mn or ⁵¹Mn (e.g. in a 1:1 ratio), it is practically possible to target the complex to as MRI imaging agent due to paramagnetic Mn (II) while ⁵²Mn or ⁵¹Mn will act as a radioisotope for PET.

Additional radioactive metals (e.g. ²²⁵Ac, ¹⁷⁷Lu, ¹¹¹In, ⁹⁹Tc or ¹⁶⁶Ho) may also be included if they would advantageously contribute to the utility of the present invention. The inventor also envisages that radiolabelling techniques, such as ¹⁸F (for PET) and ¹⁹F (for hyperpolarised MRI) may be utilised, for example by functionalisation on L-X (e.g. on a pyridine group's nitrogen atom). Such may enable simultaneously for both MRI and PET with MRI machine with a PET insert or as Standalone modalities.

In the compound of formula (I), the ligand L is an aminopolycarboxylic acid ligand, specific examples of which include ethylene diamine tetra acetic acid (EDTA), diethylene triamine penta acetic acid (DTPA), 1,4-bis(carboxymethyl)-6-[bis(carboxymethyl)]amino-6-methylperhydro-1,4-diazepine (AZTA), cyclohexylene dinitrilo tetra acetic acid (CDTA), 1,4,7,10-tetraazacyclododecane-1,4,7,10-tetraacetic acid (DOTA), nitrilotriacetic acid (NTA) and 1,2-propylenediaminetetraacetic acid (PDTA).

In some embodiments of the compound of formula (I), the ligand L may be a hexadentate or octadentate aminopolycarboxylic acid, specific examples of which are ethylene diamine tetra acetic acid (EDTA, a hexadentate ligand) or diethylene triamine penta acetic acid (DTPA, an octadentate ligand), both of which are pictured below.

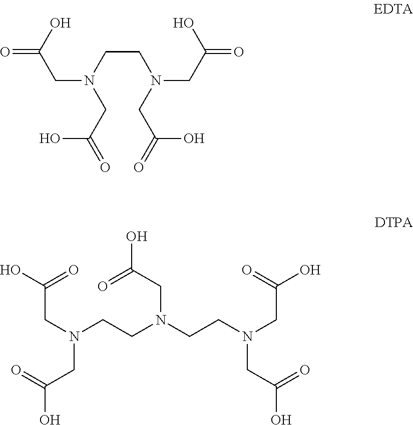 [SMILES: O=C(O)CN(CCN(CC(=O)O)CC(=O)O)CC(=O)O ; O=C(O)CN(CCN(CC(=O)O)CC(=O)O)CCN(CC(=O)O)CC(=O)O]

As would be appreciated, EDTA and DTPA may be substituted at a number of locations, including at the amine or carboxylic groups. It is also possible to substitute on one (or more) of the carbon atoms in the EDTA's or DTPA's backbone.

In some embodiments, L-X may be an EDTA-N,N″-bis(amide) substituted ligand (as shown below).

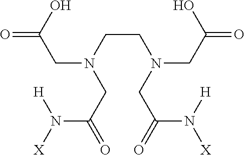

As would also be appreciated, DTPA may be substituted at a number of locations (more so than EDTA), given its higher number of amine and carboxylic acid functional groups. In some embodiments, for example, L-X may be a symmetrically or an asymmetrically monosubstituted DTPA, a symmetrically or asymmetric bis-substituted DTPA or a trisubstituted DTPA. The structural formulae for examples of such are set out below.

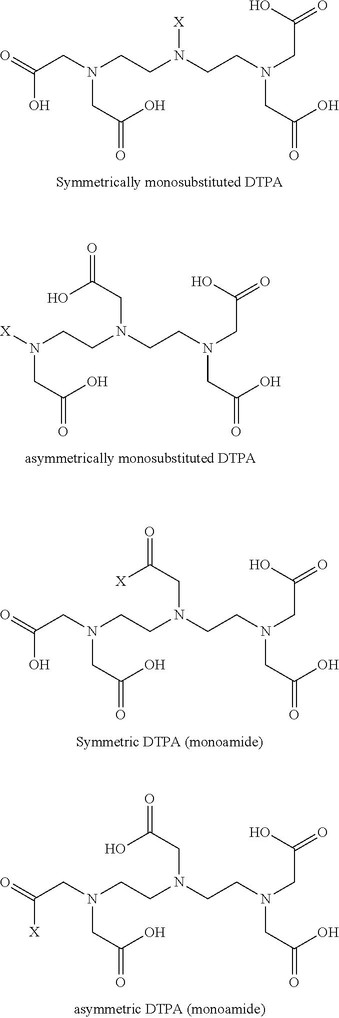

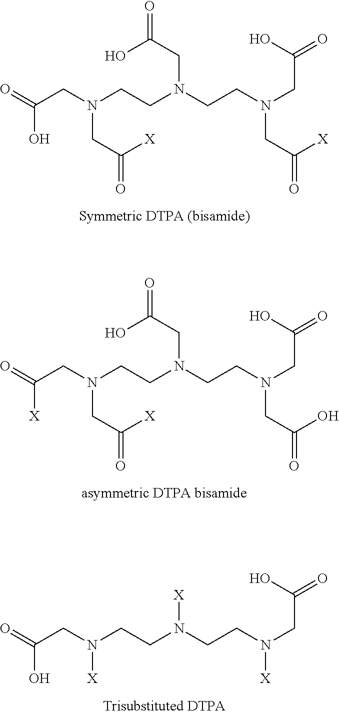

In the compound of formula (I), X is a chromophoric substituent on L that is therapeutically active. In vivo, substituent X should be cleaved from the compound of formula (I), whereupon it can realise its therapeutic effect. In some embodiments, substituent X may be released only once it has reached a target area of the patient's body (such as a cancerous growth), thereby providing a targeted therapeutic effect. As described above, for example, substituent X may be an Aurora kinase B inhibitor, which may lead to the compound of formula (I) having an anticancer activity as well as being useful as a diagnostic agent.

The compound of formula (I) may include one or more of the chromophoric substituents X, wherein each substituent X (i.e. in embodiments including a plurality of the substituents X) is the same or different. X may be substituted at any chemically possible location on L. In some embodiments, for example, X is substituted at a carboxylic group of L. In some embodiments, for example, X is substituted at an amine group of L.

The chromophoric substituent X may, for example, comprise a moiety selected from the group consisting of: 4-amino methyl pyridine (which is a Ca channel blocker/Voltage-gated K channel blocker), 2-amino anthraquinone (which has anticancer activity), sulphonamide (which is a carbonic anhydrase inhibitor), N-(2-aminoethyl)-1,8-napthalimide (which has anticancer activity), 4-aminophenol, 9-amino acridine (which has anticancer activity) and 5-amino naphthalene-2-sulphonic acid.

The chromophoric substituent X may be lumophoric and/or fluorophoric. Such incorporates modulation of luminescence (quenching or enhancement of fluorescence) upon coordination with the transition or lanthanide metal ions M. Such should facilitate their use in a variety of optical imaging modalities (not MRI and PET), both in preclinical and clinical settings. Potential use could be in fluorescence-molecular tomography (FMT) to precisely locate the surrogate marker and to quantify the same. Overlaying of the images obtained from both FMT as well as MRI and/or PET facilitates better image acquisition with high resolution while co-validating the localization of targeted probes at the specific site.

Ligand L and substituent X may be linked directly, or via any suitable chemical linking group, such as the amide linking groups described below.

In specific embodiments, L-X may be selected from the group consisting of:

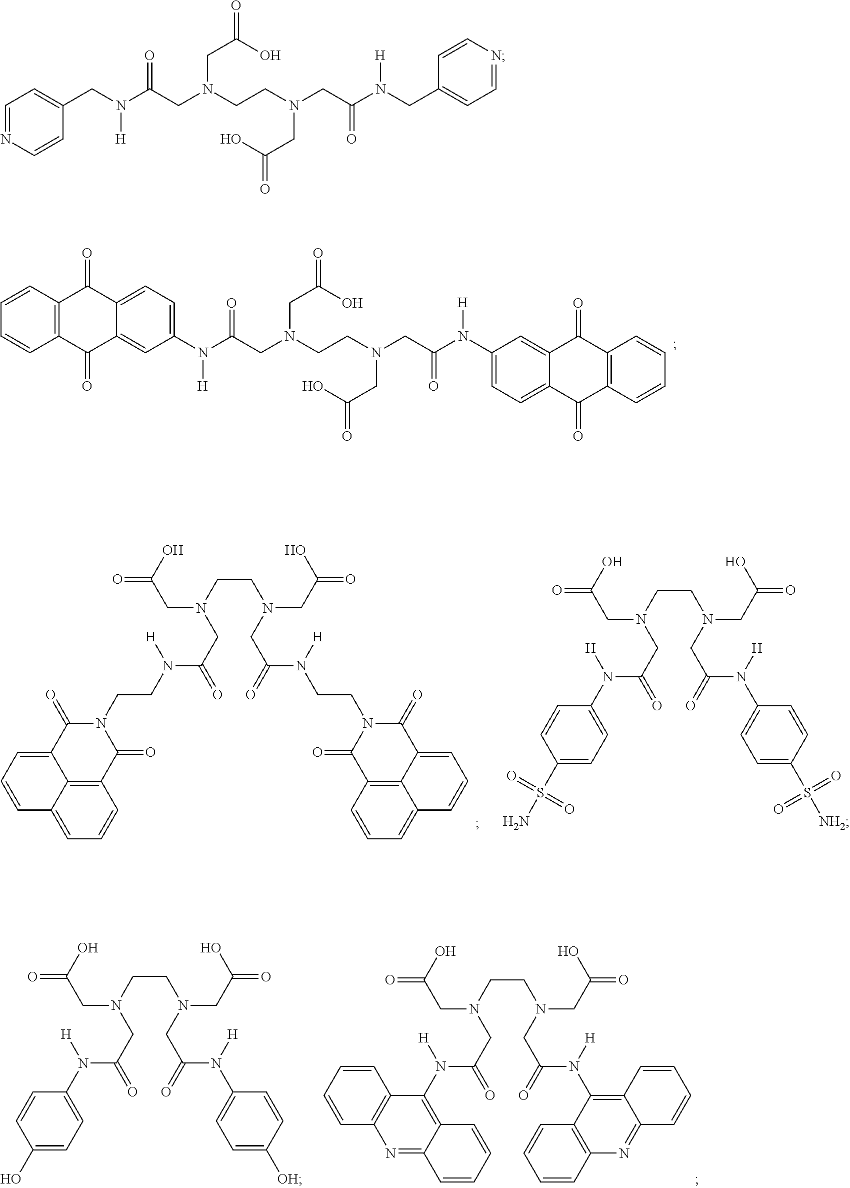

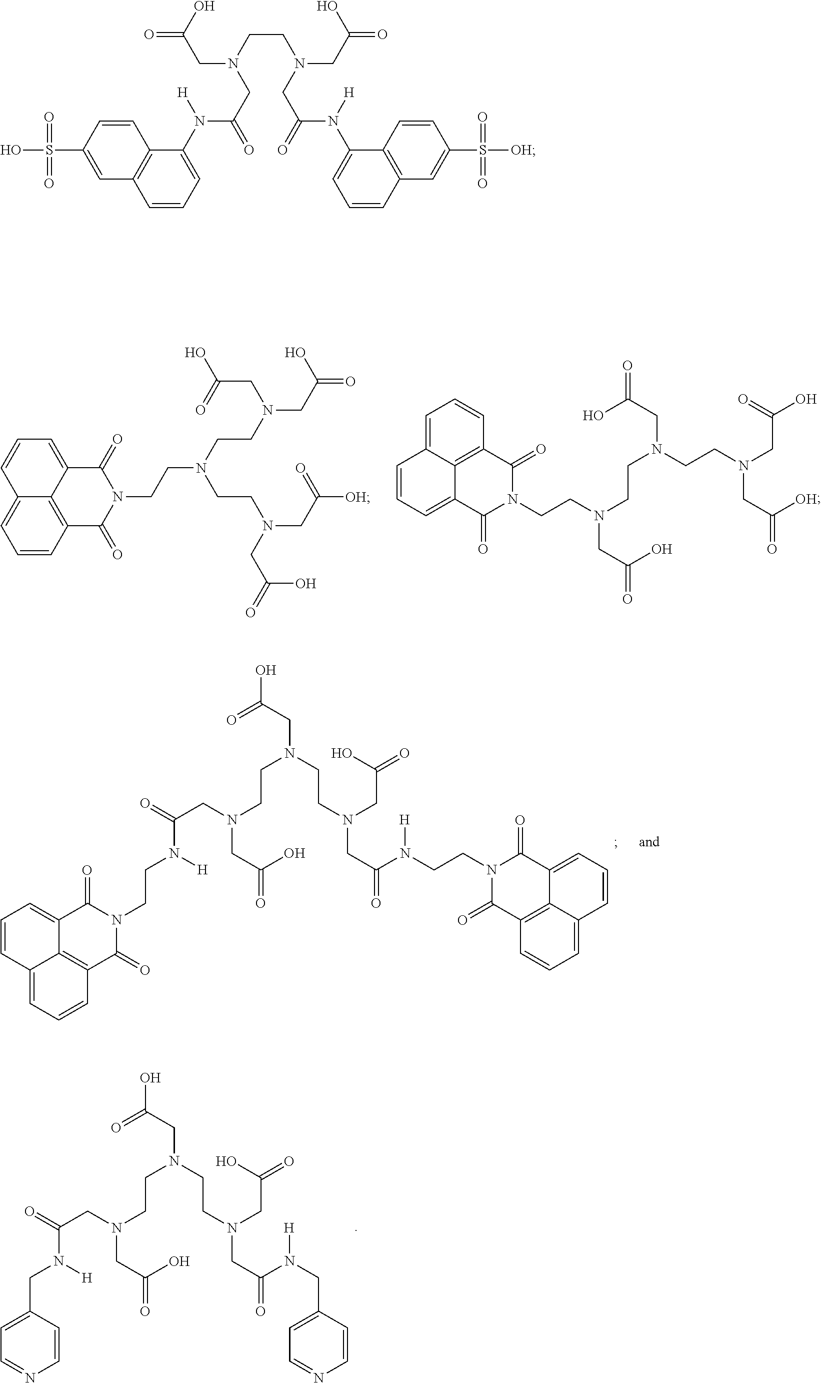

The inventor has found that a number of the compound having the formula (I) and set out above are multi-modal imaging agents.

Compounds having the formulae (I) and (II) may be synthesised using any suitable reaction scheme, specific embodiments of which will be described below. By way of general example, metal complexes of bisamide derivatives of EDTA may be formed using conventional reaction schemes such as that depicted below.

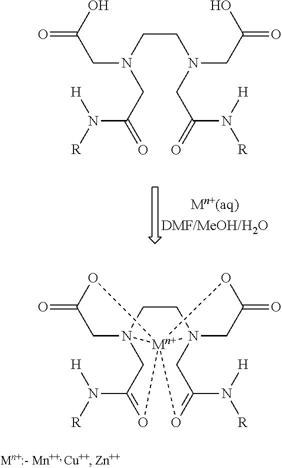

Pharmaceutical compositions including compounds having the formulae (I) and (II) which are suitable for delivery to a patient may be prepared immediately before delivery into the patient's body or may be prepared in advance and stored appropriately beforehand.

The pharmaceutical compositions and medicaments for use in the present invention may comprise a pharmaceutically acceptable carrier, adjuvant, excipient and/or diluent. The carriers, diluents, excipients and adjuvants must be “acceptable” in terms of being compatible with the other ingredients of the composition or medicament and the delivery method, and be generally not deleterious to the recipient thereof.

Compounds having the formulae (I) and (II) may also be incorporated into metal-organic frameworks and nanoparticles in order to enhance their theranostic activity. The metal-organic frameworks could, for example, be constructed by the MnL1 itself or by Zirconium oxide nanoparticles, MOF creation with Zr and EDTAMPY via a hydrothermal synthetic route, followed by encapsulation of ⁵⁵Mn/⁵²Mn(II) and subsequent coating with DOPC-based lipids and/or Albumin coating to ensure serum colloidal stability, overcoming opsonization in the form of tunable ultra-small particles (15 -50 nm) as well. Alternatively, gold-coated super ion oxide nanoparticles with pendant PEG linkers bearing nanocapsules could encapsulate MnL1 and release in vivo with tumour acidic pH.

It will be understood that, where appropriate, some of the components in the combinations or pharmaceutical compositions described herein may be provided in the form of a metabolite, pharmaceutically acceptable salt, solvate or prodrug thereof “Metabolites” of the components of the invention refer to the intermediates and products of metabolism.

“Pharmaceutically acceptable”, such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base addition salts that retain the biological effectiveness and properties of the components and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flow ability and solubility of compounds. See, e.g., H. Ansel et. al., Pharmaceutical Dosage Forms and Drug Delivery Systems (6th Ed. 1995) at pp. 196 and 14561457, which is incorporated herein by reference.

“Prodrugs” and “solvates” of some components are also contemplated. The term “prodrug” means a compound (e.g., a drug precursor) that is transformed in vivo to yield the compound required by the invention, or a metabolite, pharmaceutically acceptable salt or solvate thereof. The transformation may occur by various mechanisms (e.g., by metabolic or chemical processes). A discussion of the use of prodrugs is provided by T. Higuchi and W. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### EXPERIMENTAL RESULTS

Example 1—Synthesis of Compounds Having the Formulae (I) and (II) Where L is EDTA

Reagents were obtained from commercial sources and used as received unless otherwise stated. Solvents were dried and distilled under N₂ immediately before use. All compounds were prepared under N₂ using standard Schlenk techniques. ¹H and ¹³C[¹H]-NMR spectra were recorded on a Bruker ARX 400 spectrometer at 20° C. in D₂O or d6-DMSO. Mass spectra were performed on a micro mass Platform II system operating in Flow Injection Analysis mode with the electrospray method. Infrared spectra were recorded on a JASCO FTIR-410 spectrometer between 4000 and 600 cm⁻¹ as KBr pellets. UV/Vis spectra were recorded with a JASCO V-570 spectrophotometer.

### Synthesis of 2,2′-(3,10-dioxo-1,12-di(pyridin-4-yl)-2,5,8,11-tetraazadodecane-5,8-diyl)diacetic Acid (L1)

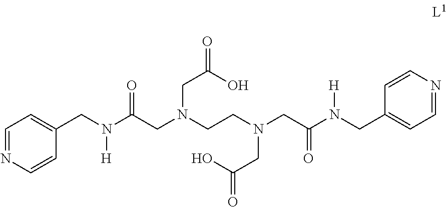 [SMILES: O=C(O)CN(CCN(CC(=O)O)CC(=O)NCc1ccncc1)CC(=O)NCc1ccncc1]

4-(aminomethyl)pyridine (0.73 g, 6.76 mmol) in DMF (10 ml) was added dropwise to EDTA bisanhydride (0.867 g, 3.38 mmol) in DMF (10 ml). The mixture was stirred overnight at room temperature. Dichloromethane (50 ml) was added to give a precipitate which was filtered and washed with acetone and acetonitrile. Yield 1.4 g (88%). ¹H NMR (400 MHz, D₂O with K₂CO₃): δ_{H}=8.30 (d, 4H, 3J_{HH}=5.8 Hz, ArH), 7.20 (d, 4H, 3J_{HH}=5.6 Hz, ArH), 4.35 (s, 4H, NHCH₂), 3.30 (s, 4H, NCH₂COOH), 3.15 (s, 4H, NCH₂CONH), 2.70 (s, 4H, NCH₂CH₂N) ppm. ¹³C—{¹H} NMR (101 MHz, D₂O with K₂CO₃): 179.3, 175.3, 149.0, 148.8, 122.6, 59.4, 58.7, 53.5, 42.0 ppm. IR (KBr disc, cm⁻¹) v=3438(br), 1712(w), 1666(vs), 1561(w). HRMS found m/z 473.2131, calculated 473.2149 for [(L1)H]⁺.

### Synthesis of 2,2′-(ethane-1,2-diylbis((2-((9,10-dioxo-9,10-dihydroanthracen-1-yl)amino)-2-oxoethyl)azanediyl)diacetic Acid (L2)

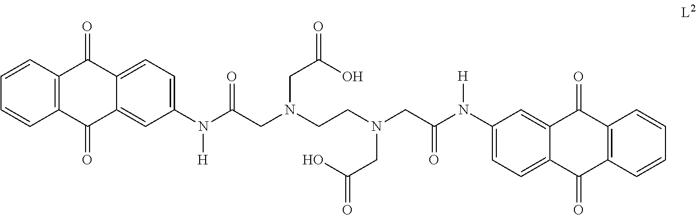 [SMILES: O=C(O)CN(CCN(CC(=O)O)CC(=O)Nc1ccc2c(c1)C(=O)c1ccccc1C2=O)CC(=O)Nc1ccc2c(c1)C(=O)c1ccccc1C2=O]

2-aminoanthraquinone (1.614 g, 7.23 mmol) in DMF (15 ml) was added dropwise to EDTA bisanhydride (0.926 g, 3.62 mmol) in DMF (15 ml). The mixture was stirred overnight at room temperature and then filtered. The filtrate was evaporated to dryness and the crude product washed with dichloromethane several times to obtain the product as dark brown glassy material. Yield 1.6 g (72%). ¹H NMR (400 MHz, d6-DMSO) δ_{H}=10.6 (s, 2H, CH₂COOH), 8.25 (s, 2H, NH), 8.09 (m, 2H, Ar), 7.95 (m, 10H, Ar), 7.75 (s, 2H, Ar), 3.6 (s, 4H, NCH₂COOH), 3.5 (s, 4H, NCH₂CONH), 2.85 (s, 4H, NCH₂CH₂N). ¹³C—{¹H} NMR (101 MHz, d6-DMSO): δc=182.3, 181.2, 173.1, 171.0, 144.3, 134.6, 134.3, 134.0, 133.1, 128.9, 127.9, 126.8, 126.7, 123.9, 116.0, 58.7, 55.8, 53.1 ppm. IR (KBr disc, cm⁻¹) v=3440(br), 1673(vs), 1624(s), 1591(vs), 1379(w), 1328(s) 1296(m), 1261(vs); HRMS found m/z 725.1837, calculated 725.1860 for [(L2) Na]⁺.

### Synthesis of 2,2′-(3,10-dioxo-1,12-disulfo-2,5,8,11 tetraazadodecane-5,8-diyl)diacetic Acid (L3)

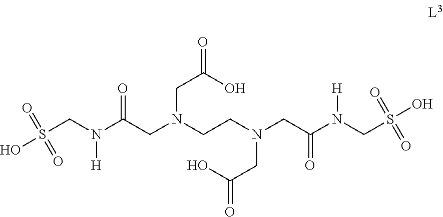 [SMILES: O=C(O)CN(CCN(CC(=O)O)CC(=O)NCS(=O)(=O)O)CC(=O)NCS(=O)(=O)O]

EDTA bisanhydride (1.8 g, 7.025 mmol) was added in small aliquots to a mixture of aminomethanesulfonic acid (1.56 g, 14 mmol) and NaHCO₃ (0.456 g, 5.43 mmol) in water (30 ml) at 0° C. The mixture was stirred for 4 h at this temperature and also at room temperature for 24 h and then filtered. The filtrate was evaporated to dryness and the crude product was washed with hot methanol. Yield 0.8 g (53%). ¹H NMR (400 MHz, D₂O) δ_{H}=4.40 (s, 4H, NHCH₂SO₃H), 3.80 (s, 8H, NCH₂COOH & NCH₂CONH), 3.50 (s, 4H, NCH₂CH₂N) ppm. ¹³C—{¹H} NMR (101 MHz, D₂O) δc=173.9, 169.6, 68. 9, 67.5, 64.6, 49.1; IR (KBr disc, cm⁻¹)v=3443(br), 3240(br), 3079(br), 1636(s), 1409(s), 1209(s), 1041(vs); ESI-MS found m/z 478.11, calculated 478.12 for [(L3)H]⁺.

### Complexation of L1, L2 and L3 with Mn(II) and Cu(II)

The in situ complexations were carried out with chlorides of Mn(II) and Cu(II), namely MnCl₂.6H₂O and CuCl₂.H₂O, for L1-L3 with 1:1 stoichiometric molar ratio for relaxometric and potentiometric titrations (described below). The schematic representation for the synthesis of the EDTA bisamides described above and their transition metal complexes is set out below. The compounds (b), (c) and (d) were used to synthesize the EDTA bisamides L1, L2 and L3, respectively.

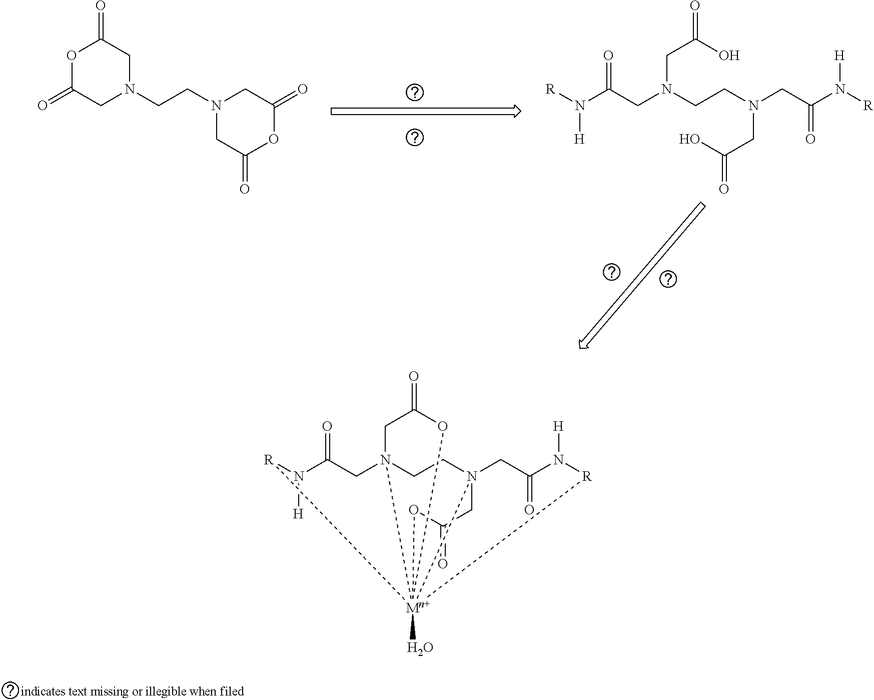

Mⁿ⁺:—Mn⁺⁺, Cu⁺⁺, Zn⁺⁺
R:—Auxilliary groups used to sythesize EDTA bisamides: (a) sulfanilamide; (b) 4-(aminomethyl)pyridine (c) 2-aminoanthraquinone and (d) aminomethanesulfonic acid

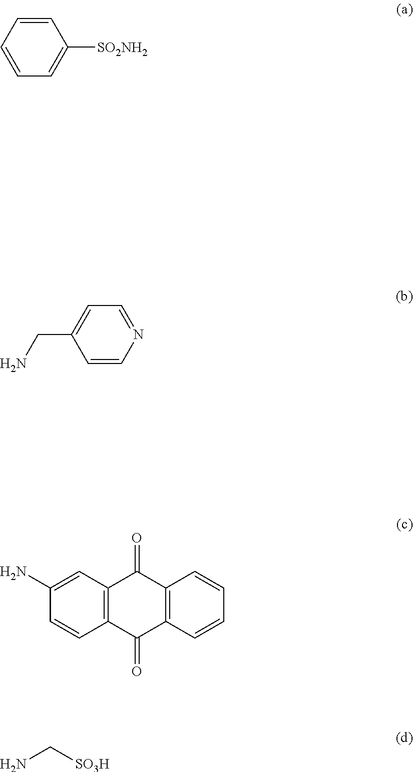 [SMILES: NS(=O)(=O)c1ccccc1 ; NCc1ccncc1 ; Nc1ccc2c(c1)C(=O)c1ccccc1C2=O ; NCS(=O)(=O)O]

### Preparation of [⁵²Mn]Mn-L1

⁵²MnCl₂ (t_{1/2}=5.6 days) in 0.3 mL aqueous HCl was obtained from the Cyclotron Facility at ANSTO. A 0.50 M solution of [⁵⁵Mn]Mn-L1 was prepared as per the published protocol for [⁵²Mn]Mn-DPDP. A 0.010 M solution of [52Mn]Mn-DPDP will be prepared as per a published protocol. A 0.30 mL aliquot of 50 mM Mn-DPDP will be spiked with 0.94 mCi ⁵²MnCl₂ in 0.030 mL aqueous HCl. The pH will be carefully adjusted to pH 3.0 by the addition of 1 M HCl and will be stirred at room temperature for 30 minutes. Subsequently, pH will be adjusted back to pH 8.0 by the addition of 1 M N-methyl-D-glucamine. The solution will be then diluted with pure water to a total volume of 1.5 mL and will be filtered through a cation exchange cartridge (Supelco Discovery™ DSC-WCX).

The final pH will be pH 8.0. [⁵²Mn]Mn-DPDP is the only ⁵²Mn containing species that will be detected by HPLC equipped with a gamma detector. The [⁵²Mn]Mn-DPDP stock is sterile and will be filtered into a sterile sealed glass vial. A similar procedure will be adapted for the preparation of [⁵²Mn]Mn-L1, however, the cation exchange cartridge will be the Grace Alltech™ IC-Chelate instead of DSC-WCX.

HPLC equipped with a gamma detector (reaction progress was monitored by radio-TLC) was used to confirm that the ⁵²Mn radiolabelled compound Mn-L1 was obtained, demonstrating that it is possible to radiolabel with ⁵²Mn, along with ⁵⁵Mn (which is paramagnetic). Spiking ⁵²MnCl₂ to ⁵⁵MnL1 complex in situ in a 1:1 ratio has shown that this generates a hybrid labelled compound, potentially enabling a nano theranostic agent delivery system targeted for chemo/radiation combined therapy. Moreover, gamma-emitting ⁵²Mn could be harnessed for its therapeutic potential.

The same procedure could be adapted for ⁵¹MnCl₂ containing the ⁵¹Mn isotope instead of ⁵²Mn.

### Example 2—Confirmation of Diagnostic Applications

The potential of L1 and L2 to be used in magnetic resonance imaging/Optical Imaging (MRI/OI) was evaluated by measuring their (a) thermodynamic stability by stability constants with potentiometric titrations; (b) R₁ relaxivities by NMR relaxivity studies and (c) spectrophotometric (luminescence) investigations.

The diagnostic moiety of the theranostic agent of the present invention enhances MR images when the patient is subjected to the magnetic field of the MR scanning machine by altering the T1 relaxation of local water protons. The paramagnetic relaxation of water protons arises as a result of dipole-dipole interactions between the proton nuclear spins and that of the oscillating magnetic field of MR machine caused by the interaction with the unpaired electron spins of paramagnetic metal ion in the complex, under pulse of radio frequency. The overall paramagnetic water proton relaxation rate enhancement (i.e. proton relaxivity) is governed by two factors, namely inner sphere and outer sphere contributions. The inner sphere contribution arises due to the interaction of paramagnetic electrons spins with that of the water protons in the first coordination sphere of the compound of formula (I) through the chemical exchange of water protons in the bulk. Apart from directly coordinated water molecules, a solvent (water) molecule could be attached to the ligand L-X or the inner-sphere of the compound via hydrogen bonding. Therefore, bulk water molecule diffusion to the paramagnetic centre of the complex of formula (I) also influences the paramagnetic effect thus contributing to the overall relaxivity. However, manipulation of inner-sphere water molecules is practically more feasible than through outer-sphere modulation. Moreover, the said complex, the functional amide group also could contribute to relaxivity enhancement by the hydrogen bonding via the C—H portion. The molecular dynamics could be best understood through the fast field cycling relaxometry, which is based on basic NMR principles. In fast field cycling (FFC), the measurement of r₁ is repeated over a range of magnetic field strengths to obtain a profile of r₁ variations as a function of the water proton NMR resonance frequency, known as nuclear magnetic relaxation dispersion profile (NMRD). This profile measures molecular dynamics taking place within the said complex understudy in a quantitative manner in the time scales of milliseconds to nanoseconds. The application of NMRD by FFC has been very recently translated into direct medical imaging in vivo of soft tissue tumours.

Although a great deal of spatial and/or temporal information could be obtained by MRI, it lacks sensitivity. The sensitivity is best achieved by optical imaging modality and/or positron emission tomography (PET). In compounds of the present invention (e.g. those incorporating L1 & L2, described above, fluorescence modulation would be expected upon coordination with the transition metal ions M via the suppression of photo-induced electron transfer mechanism. This fluorescence modulation facilitates the use of these ligands to act as photo-induced electron transfer sensors, finding application in fluorescence-based optical imaging modalities such as widefield, confocal, two-photon or multiphoton, super-resolution, and fluorescence molecular tomography (FMT) and other optical imaging-based approaches. This fluorescence modulation is reflected in the fluorescence intensity variation.

Mn-L1 was found to have a diagnostic performance comparable to the commercially available gadolinium-based contrast agents and higher than a clinically approved manganese-based contrast agent. Specifically, Mn-L1 exhibited relaxivity of 3.52 mM⁻¹s⁻¹ (30 MHz, 37° C.). Furthermore, the photophysical characterization confirmed higher Stokes shift and the ability of L1 to act as an on-off type for Cu (II). Time-resolved fluorescence investigations (TCSPC) indicated the potentiality of L1 for live-cell imaging.

The data obtained from relaxometric and potentiometric titrations illustrates the ability of Mn-L1 to serve as a potential non-gadolinium based MRI contrast agent. In addition, photophysical characterization related to L1 and L2 indicated their potential to act as fluorescent-based chemosensors, with potential applications in biology and medicine. More specifically, the ability of L1 to act as a Turn-off sensor for cupric ions in solution at room temperature along with the potential for application in live-cell imaging is justified by time-resolved fluorescence.

### Example 3—In Silico Calculations

The potential of L1 for therapeutic activity was investigated by molecular docking studies. In silico molecular modelling studies were undertaken, in which the chemical structure of L1 was drawn in ChemDraw binary format (cdx) using ChemDraw Professional 16.0 and subsequently converted to SDF file format using the Open Babel 3.1.1 tool.

Aurora B kinase inhibitory activity, along with binding characteristics of human serum albumin, of L1 was theoretically investigated by using in silico molecular docking simulation. The X-ray crystal structure of the target proteins, human Aurora B Kinase and human serum albumin (HSA) were obtained from the RCSB Protein Data Bank (PDB ID: 4AF3 and 1H9Z, respectively). The stable confirmations of the target and molecules were obtained after energy minimization using the Lamarckian genetic algorithm in Autodock vina. Prior to this, the proteins were prepared using Swiss-PDB viewer. The active sites of the target proteins were identified using the computed atlas for surface topography of proteins CASTp software. A grid with specific dimensions 25 Å×25 Å×25 Å is covered with the active site region of the target (identified by visualization in Pymol; 11e102, Phe101, His134, His135, Pro135, Pro337, Arg139, Glu155, Glu213, Tyr141, Tyr156, Lys 215, Leu 865). L1 was docked to the target protein using PyRx-Virtual screening tool version 0.8 through inbuilt Autodock vina, and their binding energies are calculated. Nine confirmations of L1 were obtained from the docking protocol, and the confirmation with the best-scored pose and with the lowest binding energy was selected for further study. BIOVIA discovery studio 3.5 (BIOVIA Discovery Studio Visualizer Software 2021), as well as Pymol software, were used to visualize the 2D and 3D representations of the intermolecular interactions between the proteins and L1.

L1 displayed a docking score of −7.5 kcal/mol with 4H-bonds interaction at distances of 2.9, 3.1, 3.2, and 3.4 A for Aurora B kinase. EDTAPA and DPDP (the ligand for TESLASCAN®), along with respective co-crystallized ligands in the form of MB4, VX-680, and R-Warfarin, were employed as controls for validation of the docking protocol, the dataset comprising the control and target compounds was docked into the active pockets of our selected targets. The results showed that L1 possessed comparable or slightly higher binding activity than its controls while it was lower than the co-crystallized respective ligands. Furthermore, validation of the binding site was carried out using the in silico binding site prediction tool CASTp 3.0 software to confirm the correct identification of active pockets in respective targets. Aurora B kinase inhibitors are well known as anti-cancer drugs causing cell death. The strong binding potential of L1 to Aurora B Kinase implies that it could be potentially explored as an anticancer agent.

In summary, these data indicate that water-soluble manganese complexes of EDTA bisamide including 4-(aminomethyl)pyridine (L1) could serve as potential non-gadolinium-based MRI contrast agent and/or PET agent. Additionally, they could act as fluorescent sensors with potential applications in biology and medicine. In silico modelling studies indicate that L1 has a strong affinity for HSA and that it may effectively inhibit Aurora B Kinase with associated anticancer activity. MnL1 and CuL1 as PET/OI imaging agents are also envisaged.

The inventor believes that these data enable a reasonable prediction to be made that compounds of formula (I) and formula (II) may have both diagnostic and therapeutic potential as theranostic agents. The inventor believes that the present invention may provide a novel nano theranostic delivery system based on L1 and a high Z element and/or Cerenkov-based light irradiation such that the system will harness the therapeutic potential of a non-therapeutic radioisotope (⁵²Mn), culminating in a novel combined chemo/radiation therapy targeted towards malignant non-small cell lung cancer (NSCLC) and/or liver cancer patient community vulnerable to radiation (brachy) and/or chemotherapy.

### Example 4—Synthesis of Compounds Having the Formula (I) Where L is DTPA

Two DTPA analogues, functionalised with a 1,8-naphthalimide chromophore (i.e. substituent X), have been successfully prepared and fully characterized. Their Gd(III) complexes have also been prepared and evaluated for their ability to act as dual modal contrast agents (MRI/OI).

The ligand contains a single organic luminescent moiety which has been directly alkylated to the nitrogen atom of a diethylene triamine. Two possibilities exist, with the lumophore being alkylated to the central nitrogen or to one of the two terminal nitrogens, as shown below.

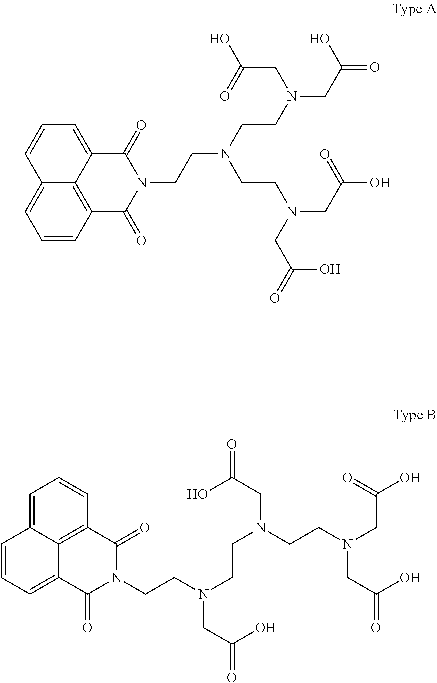 [SMILES: O=C(O)CN(CCN(CCN(CC(=O)O)CC(=O)O)CCN1C(=O)c2cccc3cccc(c23)C1=O)CC(=O)O ; O=C(O)CN(CCN(CCN1C(=O)c2cccc3cccc(c23)C1=O)CC(=O)O)CCN(CC(=O)O)CC(=O)O]

Type A (the symmetric ligand) may be synthesised via the reaction scheme set out below.

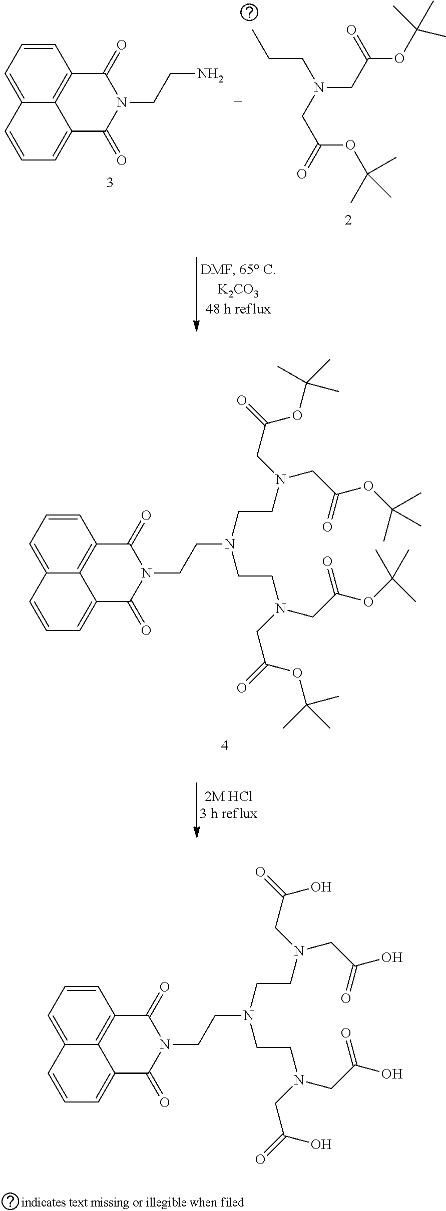 [SMILES: NCCN1C(=O)c2cccc3cccc(c23)C1=O ; CCCN(CC(=O)OC(C)(C)C)CC(=O)OC(C)(C)C ; CC(C)(C)OC(=O)CN(CCN(CCN(CC(=O)OC(C)(C)C)CC(=O)OC(C)(C)C)CCN1C(=O)c2cccc3cccc(c23)C1=O)CC(=O)OC(C)(C)C ; O=C(O)CN(CCN(CCN(CC(=O)O)CC(=O)O)CCN1C(=O)c2cccc3cccc(c23)C1=O)CC(=O)O]

Type B (the asymmetric ligand) may be synthesised via the reaction scheme set out below.

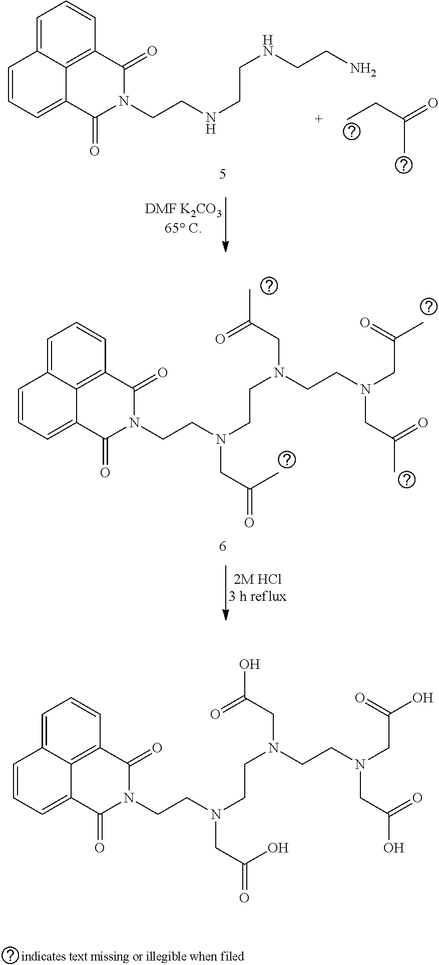

Complexation with Gd(III) was achieved using the following method. The ligand (28 mg, 0.05 mmol) and GdCl₃. 6H₂O (18.0 mg 0.05 mmol) were added to two different vials. The ligand was dissolved in ethanol 10 ml, and heated slightly to ensure complete dissolution. Then GdCl₃ was dissolved in distilled H₂O, and again heated to ensure complete dissolution. Thereafter the vial containing the metal salt solution was continuously stirred, and the ligand solution was added dropwise. The instantaneous formation of a precipitate was observed. After the complete addition of the ligand to the metal, the mixture was stirred for two days in the dark. The solvent was evaporated to give a yellow precipitate of the complex. Yield 84%; IR (KBr disc, cm⁻¹): 3425(br), 1729(w), 1625(s), 1408(s); ESI-MS (-ion): found m/z 712.0898, calc. 712.0890 for [(L1) Gd]. UV/vis [λmax, nm (ε, M−1 cm−1)] in H2O: 235(17658), 274(4128), 344(6279).

The complexation reaction of the ligands with EuCl₃, YbCl₃ and NdCl₃ were also carried out as described for complexation with GdCl₃. However, in the case of NdCl₃ it had to be dissolved in DMF, instead of water.

The Gd(III) complexes described above were found to exhibit ligand-based luminescence and to have a relatively high relaxivity. It has been reported, that for low molecular weight Gd(III) based mono aqua hydrophilic complexes, the inner-sphere and outer-sphere contributions are comparable, giving rise to relaxivity values that lie in the range of 4-5 mM⁻¹ s⁻¹ at 25° C. and 20 MHz, while the contribution from secondary-sphere water molecules has not been as extensively studied. Unusually, high relaxivity exhibited by the symmetric DTPA analogue (Type A) could be potentially due to secondary sphere water molecules. Reproducibility of the results was ensured by repeating the whole synthesis and repeating the measurements under the same conditions. Long-term reproducibility of relaxivity of the same sample, under identical experimental conditions over an extended period of time (4 months) was also established. Long term stability of the same sample in solution was also corroborated by testing with xylenol orange indicator indicating the absence of free Gd(III) in the sample. Further investigations are necessary to determine the safety profile of these reagents and the relaxivity is maintained in vivo.

Metal complexes with the monosubstituted DTPA ligands shown above are expected to be theranostic agents because 1,8 naphthalimide is a known DNA intercalating agent and would be expected to activate or inhibit DNA function and hence cure or control the spread of cancer. Furthermore, this may be achieved by the inhibition of topoisomerase I/II which causes photocatalytic DNA damage. Lanthanide complexes might also act as DNA compacting agents by the binding activity of lanthanides to DNA.

### Example 5—Prophetic Examples to Demonstrate Theranostic Activity

The inventor believes that the experiments and in silico modelling described above, in combination with the activity of known theranostic agents such as Teslascan® (Mangafodipir) enables a reasonable prediction that all compounds falling within the scope of formulae (I) and (II) may be useful as theranostic agents. In this Example, experiments which the inventor expects will use to confirm whether compounds having the formula (I) are useful as theranostic agents are described.

### Biological Evaluation

The inhibition of AURKB in tumour cells by selective AURKB inhibitors will lead to poor prognosis, thus serving as effective anticancer agents. Pyridine-based analogs have already been recognized as good AURKB inhibitors. HepG2 cancer cell lines or MCF7 cancer cell lines could be evaluated for IC50 and MTT with Doxorubicin as the reference drug.

### Aurora A Kinase In Vitro Activity Assay

The experimental method will comprise the following steps: adding 10 μL of reaction solution, 10 μL of Aurora A kinase, 10 mu L of substrate, 10 μL of solution of compound to be detected and 10 μL of LATP solution into a 96-well plate in sequence, mixing uniformly and incubating for 30 minutes. 10 μL of kinase reaction stop solution would then be added to each well plate, followed by 10 μL of phospho-histone H3 antibody in each well plate, 100 μL of LLHRP-antibody chelator solution after 60 minutes incubation at 25° C., followed by 100 μL of TMB substrate at 25° C. for 10 minutes, and finally 100 μL of ELISA stop solution in each well plate, 450 nm readings would be recorded with an ELISA detector, and IC50 will be calculated using drug-free solvent as a blank,

### In Vitro Study:

### Cytotoxicity of Compounds of Formulae (I) and (II)

The cytotoxicity of compounds of formulae (I) and (II), and its constituent moieties (including substituent X) will be examined by MTT or Suforhodamine stained cell-based assays using selected cell lines described (HepG2, MCF-7). The uptake of the free drug and lead will be examined using inductively coupled plasma mass spectrometry. The results will then form the basis for in vivo efficacy testing.

### Pharmacology-Kinetics:

### Cytotoxic Activity Assay (Anticancer Activity)

Since Aurora B kinase is abundant in hepatoblastoma (HepG2) cell lines, HepG2 cells line will be chosen as model to identify the expression effect of Aurora B kinase on the growth of hepatocellular cancer cells, in vitro. The cytotoxic activities of the prepared L-X moieties (e.g. L1 and L2) will be screened against HepG2 and BALB/3T3 (murine fibroblast) as control (normal cell line) using the standard chemotherapeutic agent doxorubicin with an IC50 value of 3.56±0.46 μg/mL. The results will be used in plotting a dose-response curve using a GraphPad prism or similar software in which the concentrations of the tested samples required to kill half of the cell population (IC50) will be determined. The cytotoxicity will be expressed as the mean IC50 and experiments will be carried out in triplicate.

### MTT Assay (Antiproliferative Assays) of Compounds of Formulae (I) and (II)

HeLa cells will be cultivated in DME (Dulbecco's Modified Eagle's culture medium), which will be supplemented with antibiotics (10⁴ units/ml of penicillin, 10 mg/ml streptomycin, and 4 mM of L-glutamine), along with 10% FBS (fetal bovine serum albumin) and will be incubated at 5% CO₂ and at 37° C. in an incubator for 48-72 h. Cell passaging will be carried out in between to make sure the culture medium is refreshed in a timely manner.

For the MTT-based cell viability assay, The HeLa cells will be seeded at the rate of 1×10⁴ Hela cells/cell in a 96-well microtiter plate, using the same medium conditions, will be incubated for 24 h. The following day, the plates will be removed from the incubator and then various concentrations of the metal complex of formulae (I) and (II) will be added to it via an automated micropipette and will be kept in the same incubator (5% CO₂ and 37° C.) for 36 h. after 36 h plate will be removed the MTT reagent conditioned to ambient temperature from storage will be added and color change (yellow to brown) will be observed. The number of viable cells will be evaluated with the help of a microplate reader, using the absorbance at 570 nm.

### Kinetic Stability of the Compounds of Formulae (I) and (II)

The presence of physiological anions could play an important role when using a complex as an MRI contrast agent in vivo. Phosphate, bicarbonate and fluoride anions can replace coordinated water molecules of the complex leading to a reduction in relaxivity in-vivo. The relaxivity measurement (at 1.41 T, 25° C. will be obtained in the presence of a higher excess concentration of these ions (1:200).

### In Vivo Efficacy Testing

All animals will be housed (with PC2 and Specific Pathogen Free (SPF) facility status) and utilized in the in vivo experiments will be subjected to a successful national animal care and ethics approval. Two cages of animals bearing six female nude mice in each cage will be used in animal studies to provide statistical significance of the outcome in, in-terms of one-way way ANOVA and t-test.

Prior to the commencement of the animal studies, in vitro efficacy will be evaluated by cytotoxicity assay, which is as follows. Briefly, cancer cells will be implanted into the animals and the xenograft tumors allowed to develop to a volume of 100 mm³. The animals will be divided into three groups comprising: control (saline), free drug and drug nanocluster. All drugs will be administered via intraperitoneal injection in saline on day 1 and tumour growth measured daily for a period of two weeks. The effectiveness of the compound of formula (I), incorporating Substituent X, will be determined by its ability to delay the growth compared with Doxorubicin (p<0.05).

### Dose Escalation and Optimization:

Female BALB/C Nude mice will be utilized to show that lead compounds of formulae (I) and (II) are physiologically compatible and non-toxic in certain dose ranges administered. Administration doses will be based on the maximum tolerated dose (MTD; defined as when the animals lose no more than 10% of their body weight in the days subsequent to drug administration) of the free drugs in similar animal models. Animal body weight will be monitored daily as a measure of the level of systemic toxicity. If little (<10%) change to body weight is observed in the animals being treated with the compound the dose will be increased in subsequent in vivo tests until an MTD is reached. Second evaluation criteria of the compound in vivo experiments will include a statistically relevant (p<0.05) reduction in side-effects/increased MTD compared with the free drugs.

### In Vitro Diagnostic Imaging—MRI

To evaluate the complex of formulae (I) and (II) as Ti brightening contrast agent, the T₁-weighted phantom MR images of the complex at four different concentrations (0.25, 0.5, 0.75, 1.00 mM) will be measured at 1.5 T by using a clinical MRI imager. A comparison of the image intensities will be compared by Image J (freely downloadable software) or AMIRA-AVIZO or similar software.

### In Vivo Diagnostic Imaging: MRI

The animal imaging research will be conducted in a biological resources imaging laboratory, utilizing a state of the art MRI Scanner (9.4 T). Female nude mice bearing orthotopic human hepatocarcinoma tumor xenografts will be scanned using T₁, T₂, and T₁*-weighted anatomic imaging sequences before and after administration of the compound of the present invention. Clear visibility of the theranostic agent following direct (intratumoral) injection will be expected. Animals will be also scanned dynamically during injection of the compound of the present invention to confirm injection and probe for rapidity of the theranostic agent's clearance.

### Investigation on Biodistribution: for MRI/PET

### Preparation of [⁵²Mn]Mn-L1 for MRI/PET

MnL1 will be prepared as described above. Mn-DPDP will be obtained from MedChem express. ⁵² MnCl₂ (t½=5.6 days) in 0.3 mL aqueous HCl will be obtained from the Cyclotron Facility at ANSTO. A 0.50 M solution of [⁵⁵Mn]Mn-L1 will be prepared as per the published protocol for [⁵²Mn]Mn-DPDP. A 0.010 M solution of [⁵²Mn]Mn-DPDP will be prepared as per a published protocol. A 0.30 mL aliquot of 50 mM Mn-DPDP will be spiked with 0.94 mCi ⁵²MnCl2 in 0.030 mL aqueous HCl. The pH will be carefully adjusted to pH 3.0 by the addition of 1 M HCl and will be stirred at room temperature for 30 minutes. Subsequently, pH will be adjusted back to pH 8.0 by the addition of 1 M N-methyl-D-glucamine. The solution will be then diluted with pure water to a total volume of 1.5 mL and will be filtered through a cation exchange cartridge (Supelco Discovery™ DSC-WCX).

The final pH will be pH 8.0. [⁵²Mn]Mn-DPDP is the only ⁵²Mn containing species that will be detected by HPLC equipped with a gamma detector. The [⁵²Mn]Mn-DPDP stock is sterile and will be filtered into a sterile sealed glass vial. A similar procedure will be adapted for the preparation of [⁵²Mn]Mn-L1, however, the cation exchange cartridge will be the Grace Alltech™ IC-Chelate instead of DSC-WCX. Alternatively, the exact procedure could be adapted for ⁵¹MnCl₂ containing ⁵¹Mn isotope will be utilized instead of ⁵²Mn.

Rats will be imaged in a 4.7 Tesla MRI scanner equipped with a PET insert (Bruker, Billerica, MA). Rats will be anesthetized with isoflurane (4% for induction, 1 to 1.5% for maintenance in medical air). Post-placement of a tail vein catheter for probe administration, rats will be positioned prone on a custom-built cradle. Rats will be kept warm using an air heater system and body temperature and respiration rate monitored by a physiological monitoring system (SA Instruments Inc., Stony Brook NY) throughout the imaging session. 0.5 M of [⁵²Mn]Mn-L₁ in sterile water and 0.4 μL g/animal body weight will be intravenously administered as a bolus via the tail vein. 0.01 M of [⁵²Mn]Mn-DPDP will be formulated at 0.01 M in sterile water and 1 μL per gram animal body weight was intravenously infused over 1 minute via tail vein. 4 -11 MBq activity will be administered to each rat.

Before the theranostic agent's injection, T₁-weighted MR images will be acquired using a 3D Fast Low Angle Shot (FLASH) sequence with the following acquisition parameters: repetition time (TR)/echo time (TE)=20 ms/3 ms, flip angle (FA)=30°, field of view (FOV)=80×65 mm², matrix size=267×200, 50 slices, slice thickness=0.8 mm, and acquisition time=3 min sec). Immediately after the Theranostic agent injection, the FLASH sequence will be repeated continuously with the PET acquisition performed simultaneously for 65 minutes. Rats will be then returned to their cages. Rats will be imaged again at 4-6 h, 3-4 d, and 7 d after injection for a period of 30, and 45 minutes, respectively.

### Biodistribution Studies:

Biodistribution studies will be performed on BALB/c mice (25-30 g). An aliquot of 2.1 mCᵢ of radiolabel will be injected [⁵²Mn]Mn-L1 in each mouse intravenously through tail vein. Six animals will be sacrificed by cardiac puncture and blood samples will be collected with the help of a syringe and radioactivity counts will be measured at 0.08 h, 0.25 h, 0.5 h, 1 h, 2 h and 4 h post-injection. Various organs (heart, lung, liver, spleen, kidney, stomach intestine and brain) will be removed after dissecting the animals and they made free from adhering tissue, will be rinsed with chilled saline, blotted to remove excess liquid, weighed, and radioactivity will be measured in each organ and the data will be expressed as percent administered dose per gram of the organ.

### Biodistribution in Variety of Tissues—MRI

The biodistribution of MnL1 will be studied in Wistar Han rats. Male rats (10 weeks age, 257-296 g body weight; n=13 per treatment group) will be restrained and administered MnL1 at 0, 0.15 or 0.30 mmol Gd/kg as a single intravenous bolus injection. Standard toxicology endpoints will be included in this study, including clinical toxicity, body weights, clinical pathology (clinical chemistry, hematology and coagulation) and macro-and microscopic pathology. Animals will be euthanized at Day 4 (n=7/dose level) or Day 28 (n=6/dose level) post-administration of MnL1 for complete necropsies. Selected tissues (bone, brain, kidney, liver, skin and bone) will be collected for histopathological analysis and determination of Mn levels. For determination of Mn levels, tissue samples will be frozen immediately in liquid nitrogen and stored at −20° C. until analysis.

Mn concentration in tissue samples will be quantified using inductively-coupled plasma mass spectrometry (ICP-MS). Wet tissue (Ca. 100 mg) will be digested in 90% concentrated HNO₃ (Ca. 750 μL) at 90° C. for 10-15 min. The digested sample will be diluted in deionized (DI) water, vortexed vigorously using a hand vortexer, and centrifuged at 3500 rpm for 15 min. The supernatant will be separated, and further dilution will be carried out as necessary as needed to ensure Mn concentrations fell within the range of calibration standards (1-500 ppb). Appropriate Quality control samples (50 and 100 ppb) will be included at the start, middle, and end of analysis runs.

### Pharmacokinetic Studies—MRI

The pharmacokinetics (PK) of Mn-L1 will be evaluated in cynomolgus monkeys. Briefly, non-naïve male cynomolgus monkeys (n=3 per treatment group, 2-5 yr. age, 2.3-3.1 kg body weight; Charles River Laboratories, Reno, NV) will be chair-restrained and intravenously administered Mn-L1 using a calibrated infusion pump over ˜60 min at 0.30 mmol Mn/kg. Blood samples will be collected from all animals pre-dose, immediately post-end of infusion, and 4, 8, 24, 48, 96, 168, 336, and 672 h post-start of infusion (SOI). Blood samples will be processed to plasma and stored frozen until ready for analysis. This will be a non-necropsy study and the assessment of Mn-L1 toxicity will be limited to clinical toxicity, body weights, and clinical pathology (clinical chemistry, hematology, and coagulation) measurements. Animals will be returned to the laboratory colony at the termination of the study.

Mn concentration in plasma samples will be determined using ICP-MS (Agilent, CA, USA). Plasma samples (100 μL) will be digested in 90% concentrated HNO₃ (750 μL) at 90° C. for 15 min. The digested samples will be diluted in deionized (DI) water, centrifuged at 3000 rpm for 15 min and the supernatant was further diluted for ICP-MS analysis such that the Mn concentrations falls within the range of ICP-MS calibration standards (1-500 ppb).

### In Vivo Imaging—Optical Imaging

HepG2, MCF-7 cells will be grown into subconfluency and then will be detached with trypsin, centrifuged, supernatant will be discarded, and cells will be resuspended in 0.5% BSA (Fisher Biotech) in sterile PBS (GIBCO) at 1×10⁸ cells ml⁻¹. Cells (1-4×10⁶ per spot) will be injected subcutaneously (systemic administration to support multimodal imaging) at the indicated locations on 4-8-week-old male BALB/c nude mice under isoflurane anesthesia. 2-4 weeks after cell implantation, probe (25 nmol; MnL₁) will be dissolved in 66% DMSO in PBS at a total volume of 100 ml will be injected intravenously via the tail vein to tumor-bearing mice. Mice anesthetized with isoflurane will be imaged at various time points after probe injection using the IVIS 200 imaging system (Xenogen). Relative fluorescence of equal-sized areas of tumour and back will be measured using Living Image (Xenogen). After the last time point of imaging, mice will be anesthetized with isoflurane and sacrificed by cervical dislocation. Tumors, liver and kidney, spleen and muscle, or brain will be surgically excised and frozen in liquid nitrogen. Organs will be either lysed using a bead beater, pounced in PBS buffer, pH 7.2 (1% Triton X-100, 0.1% SDS, 0.5% sodium deoxycholate, 0.2% sodium azide), or imaged ex vivo using the IVIS 200 imaging system before lysis of tissues. Total protein extracts will be separated by SDS-PAGE and will be visualized by scanning of the gel with a suitable laser scanner. The intensity of bands will be measured using Image J software or similar software. For inhibitor studies, compounds will be injected intraperitoneally twice daily in 40% DMSO/sterile PBS in a final volume of 100 ml.

As described herein, the present invention provides the use of compounds having the formulae (I) and (II) as theranostic agents. Embodiments of the present invention provide a number of advantages over existing therapies, some of which are described above.

It will be understood to persons skilled in the art of the invention that many modifications may be made without departing from the spirit and scope of the invention. All such modifications are intended to fall within the scope of the following claims.

It will be also understood that while the preceding description refers to specific forms of the compounds, pharmaceutical compositions, uses and methods of treatment, such detail is provided for illustrative purposes only and is not intended to limit the scope of the present invention in any way.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. The use of a compound having the formula (I):
M-L-X (I)
as a theranostic agent, wherein:
M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II);
L is an aminopolycarboxylic acid ligand; and
X is a chromophoric substituent on L that has a therapeutic activity.

2. The use of claim 1, wherein the substituent X is an Aurora kinase B inhibitor.

3. The use of claim 1, wherein the substituent X has an anticancer activity.

4. The use of claim 1, wherein the substituent X has an anticancer activity in relation to one or more of the following cancers: lung cancer, non-small cell lung cancer, breast cancer, bladder cancer, blood cancer, gastric cancer, ovarian cancer, liver cancer, pancreatic cancer, testicular cancer, prostate cancer, brain cancer, head/neck cancers and neuroendocrine tumours.

5. The use of claim 1, wherein the compound of formula (I) comprises a plurality of the substituents X, wherein each substituent X is the same or different.

6. The use of claim 1, wherein the substituent X is substituted at a carboxylic or an amine group of L.

7. The use of claim 1, wherein the substituent X is lumophoric or fluorophoric.

8. The use of claim 1, wherein the substituent X comprises a moiety selected from the group consisting of: 4-amino methyl pyridine, 2-amino anthraquinone, sulphonamide, N-(2-aminoethyl)-1,8-napthalimide, 4-aminophenol, 9-amino acridine and 5-amino naphthalene-2-sulphonic acid.

9. The use of claim 1, wherein L is a hexadentate or octadentate aminopolycarboxylic acid.

10. The use of claim 1, wherein L is selected from the group consisting of: ethylene diamine tetra acetic acid (EDTA), diethylene triamine penta acetic acid (DTPA), 1,4-bis(carboxymethyl)-6-[bis(carboxymethyl)]amino-6-methylperhydro-1,4-diazepine (AZTA), cyclohexylene dinitrilo tetra acetic acid (CDTA), 1,4,7,10-tetraazacyclododecane-1,4,7,10-tetraacetic acid (DOTA), nitrilotriacetic acid (NTA) and 1,2-propylenediaminetetraacetic acid (PDTA).

11. The use of claim 1, wherein L-X is selected from the group of ligands consisting of:

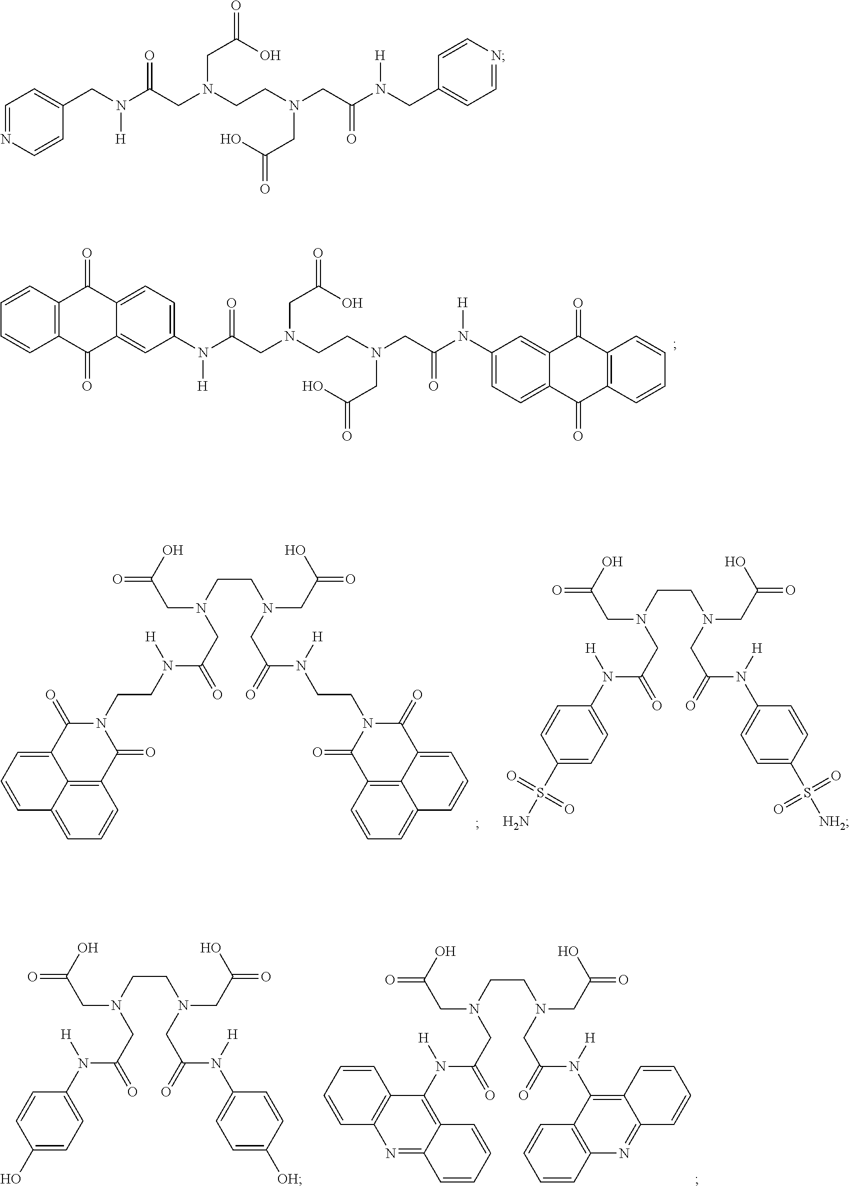

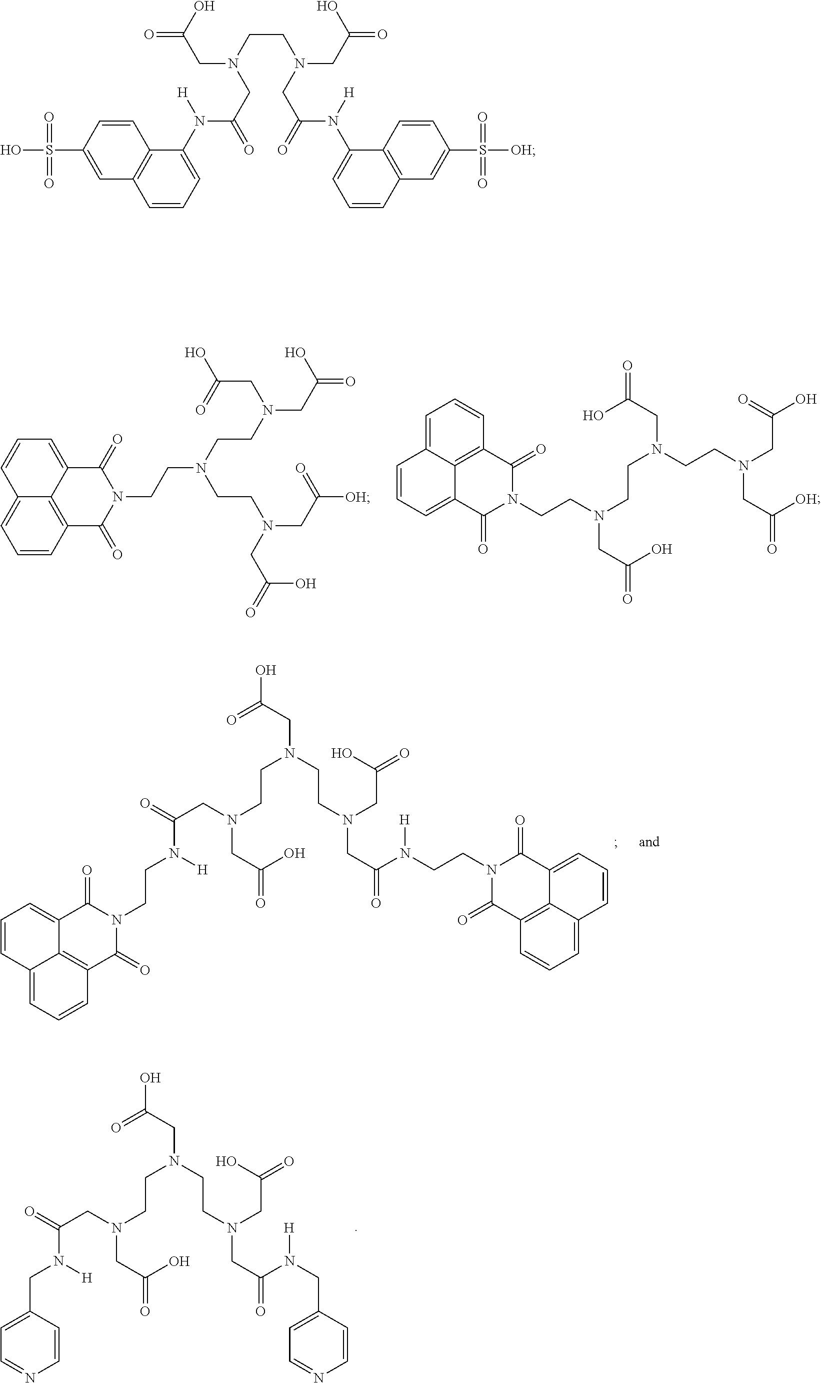

12. The use of claim 1, wherein the compound having the formula (I) is a multi-modal imaging agent.

13. The use of claim 1, wherein the compound having the formula (I) is a dual mode imaging agent.

14. The use of claim 12, wherein the compound having the formula (I) is an imaging agent for two or more of the following imaging techniques: MRI, PET, CT, SPECT Dosimetry, FLI, CLI and OI.

15. The use of claim 1, wherein M comprises a radioactive isotope of Mn(II), Cu(II), Zn(II), Gd(III), Ga(III), Zr(IV), Ac(III), Tc(IV) or Pb(II).

16. The use of claim 15, wherein M is ⁵¹Mn, ⁵²Mn, ⁶⁸Ga, ⁹⁹Tc, ²²⁵AC, ⁸⁹Zr or ²¹²Pb.

17. The use of a compound having the formula (II):
M-L (II)
as a theranostic agent, wherein:
M is a metal selected from the group consisting of: Mn(II), Cu(II), Zn(II), Gd(III), Ga(III), Eu(III), Yb(III), Nd(III), Fe(III), Tb(III), Lu(III), Zr(IV), Ac(III), Tc(IV) and Pb(II); and
L is selected from the group consisting of:

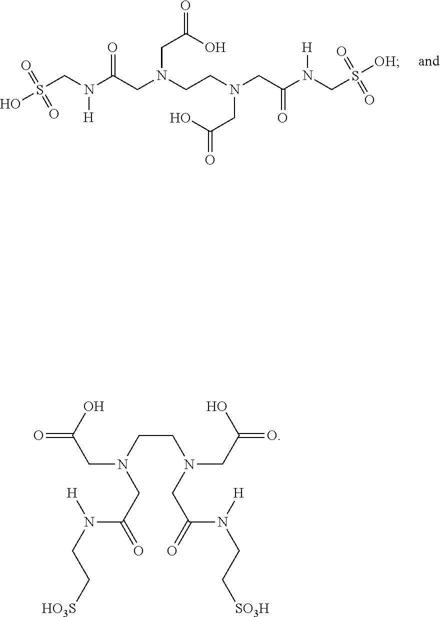 [SMILES: O=C(O)CN(CCN(CC(=O)O)CC(=O)NCS(=O)(=O)O)CC(=O)NCS(=O)(=O)O ; O=C(O)CN(CCN(CC(=O)O)CC(=O)NCCS(=O)(=O)O)CC(=O)NCCS(=O)(=O)O]

18. A method for treating cancer in a patient, the method comprising: administering a compound having the formula (I) as defined in claim 1 or having the formula (II) as defined in claim 17 to the patient.

19. The method of claim 18, further comprising imaging the cancer after administration of the compound.

20. The method of claim 18, wherein the cancer is selected from one or more of the group consisting of: lung cancer, non-small cell lung cancer, breast cancer, bladder cancer, blood cancer, gastric cancer, ovarian cancer, liver cancer, pancreatic cancer, testicular cancer, prostate cancer, brain cancer, head/neck cancers and neuroendocrine tumours.
